(19) United States
(12) Patent Application Publication
(10) Pub. No.: US 2003/0077337 A1
(43) Pub. Date: Apr. 24, 2003

(54) Synthetic layered silicate exhibiting tolerance to electrolytes in applications thereof

(76) Inventors: David B. Shaw, Old Hall (GB); Terence Timperley, Widnes (GB); Mohammed Jameel Barlas, Blackburn (GB); Jane Doyle, Chapel Lane (GB)

Correspondence Address: ERIC B. MEYERTONS, CONLEY, ROSE & TAYON, P.C., P.O. BOX 398, AUSTIN, TX, 78767-0398, US

(21) Appl. No.: 09/941,168

(22) Filed: Aug. 28, 2001

(51) Int. Cl. A61K 33/12; A61K 7/16; C01B 33/24

(52) U.S. Cl. 424/683; 423/331

(57) ABSTRACT

A synthetic layered silicate material may be produced which exhibits tolerance to high levels of electrolytes and surfactants as measured by viscosity, hydration rate and clarity. The synthetic layered silicate material may produce similar or improved rheological properties when compared to natural hectorite at comparable or lower addition rates in select aqueous systems.

### BACKGROUND OF THE INVENTION

1. Field of the Invention

The present invention generally relates to synthesis of synthetic layered silicates. More particularly, this invention generally relates to the production of a synthetic layered silicate that may develop acceptable rheological properties in aqueous solutions in the presence of relatively high levels of electrolytes or surfactants.

2. Description of the Related Art

The name “hectorite” has been ascribed to a natural trioctahedral smectite found at Hector, Calif., U.S.A. The synthesis of synthetic layered silicates similar to natural hectorite has been described by Granquist and Pollack in “Clays and Clay Minerals”, Volume 8, Proceedings of the 8th National Conference on Clays and Clay Minerals, Pages 150-169, which is incorporated herein by reference. In the process described by Granquist, gels of magnesium hydroxide and of silica are produced separately, washed, and combined and redispersed in water to form a suspension. Lithium hydroxide or lithium fluoride and sodium hydroxide are added to the suspension which is then treated hydrothermally by refluxing the suspension with stirring until a product having a crystal structure similar to that of hectorite is formed.

While synthetically produced layered silicates may have a crystal structure similar to natural hectorite, they may not have good rheological properties. Measuring the Bingham Yield Value of an aqueous dispersion of a substance may provide a standard comparator of the rheological properties between substances. The term Bingham Yield Value, also known as Bingham Yield Stress, is referred to in standard works on rheology. References to Bingham Yield Value may be found in “Rheology Theory and Applications”, F. R. Eirich, Acad. Press, Volume 1, 1956, page 658; “Colloidal Dispersions”, L. K. Fischer, N. Y. Bureau of Standards, 2nd Edition, 1953, pages 150-170 and “The Chemistry and Physics of Clays and other Ceramic Materials”, 3rd Edition, page 463, A. B. Searle and R. W. Grimshaw, all of which are incorporated herein by reference.

The Bingham Yield Value may be determined by first obtaining a flow curve relating the shear stress to the rate of shear. The straight-line section of the curve is extrapolated to the shear stress axis. The intercept at the shear stress axis is the Bingham Yield Value. The Bingham Yield Value may conveniently be determined on any viscometer capable of measuring a range of shear rates and shear stresses.

Some synthetic layered silicates, when dispersed in amounts of about 2 grams of silicate in 100 ml of tap water, may produce a Bingham Yield Value of only about 15 dynes/cm². This Bingham Yield Value may be lower than the Bingham Yield Value produced by natural hectorite at similar addition rates. The static gel strength produced by the synthetic layered silicates may also be lower than the static gel strength produced by natural hectorite.

Processes for the production of synthetic layered silicates having a crystal structure similar to natural hectorite, but which may have better rheological properties in tap water than natural hectorite have been described in British Patent Nos. 1,054,111; 1,213,122; 1,432,770 and U.S. Pat. Nos. 3,586,478; 3,671,190; 3,954,943 and 4,049,780, all of which are incorporated herein by reference. These processes describe the production of synthetic layered silicates having a characteristic hectorite-like structure and having varying contents of lithium and fluorine. The synthetic layered silicates may be produced by a process involving direct co-precipitation of a magnesium silicate.

British Patent No. 1,054,111 describes a process for producing a synthetic clay product. Magnesium ions, silicon (as silicate), hydroxyl, and sodium ions are added to a heated and stirred aqueous solution of lithium and fluoride ions. The co-precipitation reaction forms a slurry. The resulting precipitate is treated by heating the solution to reflux. The concentration of the product in the slurry is from 1% to 8% by weight. The synthetic layered silicate includes fluorine and lithium.

The process described in British Patent No. 1,213,122 involves precipitating a magnesium silicate by combining an aqueous solution of a water soluble magnesium salt with an aqueous alkaline solution of one or more sodium compounds in the presence of a dissolved silicon compound, and heating the precipitate under pressure. The concentration of the precipitate is preferably not more than 5% by weight. The synthetic layered silicate product includes no fluoride ions and optionally includes lithium ions.

The process described in British Patent No. 1,432,770 involves precipitating a magnesium silicate by combining an aqueous solution of a water soluble magnesium salt with an aqueous sodium carbonate solution, followed by addition of a dissolved silicon compound and heating the precipitate. The concentration of the precipitate is preferably 5% to 10% by weight. The synthetic layered silicate product may include either lithium or fluorine or both.

The products of the processes of the three British patents set forth above, are also characterized by producing dispersions giving exceedingly high Bingham Yield Values in excess of any known to be produced by natural hectorite dispersions and usually above 40, for example from about 50 to 250 dynes/cm² as a 2% by weight dispersion in tap water, and high static gel strength.

Certain products may produce acceptable viscosities in tap water, but it may be desirable to produce a synthetic layered silicate with acceptable rheological properties in aqueous solutions containing higher levels of electrolytes than tap water.

### SUMMARY OF THE INVENTION

In an embodiment, the synthesis of a synthetic layered silicate may be produced having a crystal structure similar to hectorite and having the general formula:

[Si₈(MgₐLi_{b})O₂₀(OH)_{4-y}F_{y}]^{z-}zM⁺

wherein a=4.75 to 5.45; b=0.25 to 1.25; y=0 to <4; z=12-2a-b; and M is Na⁺ or Li⁺.

In an embodiment, a lithium compound may be added to water followed by addition of a water-soluble magnesium compound. The aqueous mixture may be heated to above ambient temperature and gently stirred. A soluble carbonate solution may be added to the lithium and magnesium mixture with stirring. Magnesium carbonate may begin to precipitate. Subsequently, a soluble silicate solution may be added to the mixture. The resulting solution and precipitate may be stirred and hydrothermally treated for a selected time period to produce suitable crystal growth. Hydrothermal treatment may be defined as heating the solution to at least the boiling point of the solution, capturing the vapor, condensing the vapor and returning the condensate to the solution. An autoclave may be used in hydrothermal treatment. The resulting precipitate may be filtered, dried, and ground.

In an embodiment, a magnesium compound may be dissolved in water. An aqueous solution of a carbonate compound may be added to the magnesium compound. Magnesium carbonate may begin to precipitate. Compounds providing sources of lithium and/or fluoride ions may be added to the magnesium carbonate suspension. A silicate solution may be added to the magnesium carbonate suspension. The resulting solution and precipitate may be stirred and hydrothermally treated for a selected time period to produce suitable crystal growth. An autoclave may be used in hydrothermal treatment. The resulting precipitate may be filtered, dried, and ground.

Other embodiments are set forth in the description below or in the claims.

### BRIEF DESCRIPTION OF THE DRAWINGS

FIG. 1 is a graph showing the electrolyte tolerance of a synthetic layered silicate material produced by an embodiment.

### DETAILED DESCRIPTION OF THE INVENTION

In an embodiment, a synthetic layered silicate may be synthesized having the following formula:

[Si₈(MgₐLi_{b})O₂₀(OH)_{4-y}F_{y}]^{z-}zM⁺

wherein a=4.75 to 5.45; b=0.25 to 1.25; y=0 to <4; z=12-2a-b; and M is Na⁺ or Li⁺.

A water-soluble lithium compound may be added to an amount of water. A water-soluble lithium compound may be any soluble or slightly soluble lithium compound that provides a source of lithium cation when dispersed in an aqueous media. Examples of lithium compounds include, but are not limited to, lithium carbonate, lithium chloride, lithium bromide, lithium fluoride, lithium sulfate, or mixtures thereof. An excess of lithium ion content in the reaction mixture may be important in producing a synthetic layered silicate with improved electrolyte tolerances. The lithium compound may be added in amounts such that the lithium content in solution may be from about 100% to about 300% above the value of the lithium content required to provide the cation of the layered silicate. The lithium cations may be incorporated into the lattice structure of the produced layered silicate. In one embodiment, the lithium content in the produced synthetic layered silicate may be from about 0.3% to about 2.2%. In an embodiment, the lithium content, in the produced synthetic layered silicate, may be from about 0.35% to about 1.0%. In other embodiments, the lithium content, in the produced synthetic layered silicate, may be from about 0.5% to about 0.85%.

Various amounts of reactants, expressed as ranges of atomic ratios, may be used to produce a synthetic layered silicate. An atomic ratio relative to lithium, wherein lithium is 1, may be about 1-10 atoms silicon, about 1-8 atoms magnesium and about 2-16 atoms sodium. In an embodiment, the atomic ratio may be about 2-5 atoms silicon, about 1-6 atoms magnesium, and about 2-14 atoms sodium, all with respect to lithium. In an embodiment, the atomic ratio may be about 3-5 atoms silicon, about 1-4 atoms magnesium, and about 2-7 atoms sodium, all with respect to lithium. Experimental results indicate that a synthetic layered silicate produced using atomic ratios in the aforementioned atomic ratios exhibit improved tolerance to electrolytes during use. Electrolytic tolerance may be measured by adding the synthetic layered silicate to an electrolytic solution and determining the viscosity of the solution. An improved tolerance to electrolytes is characterized by a viscosity greater than 100,000 centipoise (cps) in the presence of up to about 10 meqs. of electrolyte per gram of the synthetic layered silicate.

The lithium compound may be stirred in the aqueous medium up to about 2 hours at about ambient temperature. The solution may be stirred during the reaction period at rates below about 1000 rpm, however, in other embodiments, the stirring rate may be below about 200 rpm. An additional amount of water may be added to the lithium mixture, followed by the addition of a water-soluble magnesium compound. The magnesium compound may be any water-soluble magnesium compound that may provide a source of magnesium cations when dispersed in an aqueous media. Examples of magnesium compounds include, but are not limited to, magnesium sulfate heptahydrate, magnesium chloride, magnesium nitrate, magnesium bromide, or mixtures thereof. The mixture may then be heated to greater than ambient temperature.

In a separate container, a solution of a water-soluble carbonate compound may be prepared. The water-soluble carbonate compound may be any carbonate compound that may provide a source of carbonate anions when dispersed in an aqueous media. Examples of carbonate compounds include, but are not limited to, sodium carbonate, potassium carbonate, lithium carbonate, or mixtures thereof. The carbonate solution may be added to the aqueous mixture of the magnesium and lithium compounds. The carbonate solution may be added over a period of up to about 1 hour. The temperature may be maintained at greater than about 25° C. during addition. A silicate solution may be added to the magnesium, lithium, and carbonate solution.

The silicate solution may be added to the above mixture over a period of greater than about 30 minutes, while maintaining the mixture temperature at greater than about 25° C. The silicate solution may be any solution which provides SiO₂. The silicate solution may be added to the magnesium, lithium, and carbonate solution such that, in the produced synthetic layered silicate, in an embodiment, the ratio of SiO₂/MgO may be from about 2.20 to about 2.50, however, in other embodiments the ratio may be from about 2.25 to about 2.40. In some embodiments, the SiO₂/MgO ratio may be from about 2.3 to about 2.35. Examples of silicate solutions include, but are not limited to, aqueous mixtures of sodium silicate, silicic acid, lithium silicate compounds, potassium silicate compounds, mixtures of silicon dioxide and sodium oxide solutions (i.e. water glass), or mixtures thereof.

The solution and product may be heated to the point wherein a vapor may be produced, the vapor condensed, and returned to the solution. The heating procedure may continue for longer than about 1 hour. Subsequently, the solution may be heated to greater than about 100° C. for a period greater than about 2 hours. An autoclave may be used. The resulting hydrous magnesium silicate may be separated by filtration, and subsequently washed and dried.

In an embodiment, other orders of addition of reactants or other reactants may be utilized to produce a layered silicate compound. The same types of solutions as in the aforementioned embodiments may be utilized. An aqueous solution of a carbonate compound may be added to an aqueous solution of a magnesium compound. The carbonate compound solution may be added to the magnesium compound solution over a time period greater than about 30 minutes. The reaction solution may be maintained at a temperature greater than about 25° C. The solution may be stirred during the reaction period at rates below about 1000 rpm. In other embodiments, the stirring rate may be below about 200 rpm. Magnesium carbonate may be subsequently produced. A source of lithium and/or fluoride ions may be added to the stirred suspension of magnesium carbonate. Fluoride compounds, as the source of the fluoride ion, include, but are not limited to, lithium fluoride, sodium hexafluorosilicate, hydrofluoric acid, sodium fluoride, potassium fluoride, or mixtures thereof. An excess of lithium ion content in the reaction mixture may be important in producing a synthetic layered silicate with improved electrolyte tolerances. The lithium compound may be added in amounts such that the lithium content in solution may be from about 100% to about 300% above the value of the lithium content required to provide the cation of the layered silicate. The lithium cations may be incorporated into the lattice structure of the produced layered silicate. The lithium content, in the produced synthetic layered silicate may, in some embodiments, be from about 0.3% to about 2.2%. In an embodiment, the lithium content, in the produced synthetic layered silicate, may be about 0.35% to about 1.0%, and in other embodiments, the lithium content, in the produced synthetic layered silicate, may be about 0.5% to about 0.85%.

A silicate solution, in some embodiments, may be added to the suspension of the precipitated magnesium carbonate such that, in the produced synthetic layered silicate, the ratio of SiO₂/MgO may be from about 2.20 to about 2.50. In some embodiments, the SiO₂/MgO ratio may be about 2.25 to about 2.40. In other embodiments, the SiO₂/MgO ratio may be about 2.3 to about 2.35. Examples of silicate solutions include, but are not limited to, aqueous mixtures of sodium silicate, silicic acid, lithium silicate compounds, potassium silicate compounds, mixtures of silicon dioxide and sodium oxide solutions (i.e., water glass), or mixtures thereof.

In an embodiment, the entire aqueous suspension from which the precipitate may be formed may be subjected to a hydrothermal treatment. The hydrothermal treatment may be conducted such that the entire aqueous suspension may be heated for a period of greater than about 1 hour at temperatures greater than about 100° C. An autoclave may be used. The material may be filtered, washed, and dried.

In an embodiment, a carbonate solution may be added to the magnesium solution over a period of greater than about 30 minutes. A silicate solution may be added to the magnesium and carbonate solution, followed by the addition of the lithium and/or fluoride compounds. In an embodiment, the amount of silicate solution added to the suspension of the precipitated magnesium carbonate may be such, that in the produced synthetic layered silicate, the ratio of SiO₂/MgO may be from about 2.20 to about 2.50. In an embodiment, the ratio of SiO₂/MgO, in the produced synthetic layered silicate, may be about 2.25 to about 2.40. In other embodiments, the SiO₂/MgO ratio, in the produced synthetic layered silicate, may be about 2.3 to about 2.35. The lithium compound may be added in amounts such that the lithium content in solution may be from about 100% to about 300% above the value of the lithium content required to provide the cation of the layered silicate. The lithium content, in the produced synthetic layered silicate, in some embodiments, may be from about 0.3% to about 2.2%. In an embodiment, the lithium content, in the produced synthetic layered silicate, may be about 0.35% to about 1.0%. In other embodiments, the lithium content, in the produced synthetic layered silicate may be about 0.5% to about 0.85%. The reaction solution may be maintained at a temperature greater than about 25° C. The solution may be stirred during the reaction period at rates below about 1000 rpm and, in other embodiments, at rates below about 200 rpm. The entire aqueous suspension in which the precipitate may be formed may be subjected to a hydrothermal treatment. The hydrothermal treatment may be conducted such that the entire aqueous suspension may be heated for a period of greater than about 1 hour at temperatures greater than about 100° C. An autoclave may be used. The material produced may be filtered, washed, and dried.

A synthetic layered silicate may be prepared using magnesium carbonate as a starting material, rather than generating magnesium carbonate during the manufacturing process. In an embodiment, a water-soluble lithium compound may be added to an amount of water under the same conditions as previously described. The magnesium carbonate may be added to the lithium compound solution. A silicate solution amount may be added such that the SiO₂/MgO ratio, in the produced synthetic layered silicate, may be from about 2.20 to about 2.50. In an embodiment, the silicate solution amount may be added such that the SiO₂/MgO ratio, in the produced synthetic layered silicate, may be from about 2.25 to about 2.40. In other embodiments, the silicate solution amount may be added such that the SiO₂/MgO ratio, in the produced synthetic layered silicate, may be from about 2.3 to about 2.35. The mixture may be processed as described herein.

In an embodiment, an acid toilet bowl cleaner may be produced with a synthetic layered silicate as described herein. An acid toilet bowl cleaner formulation may include water, a synthetic layered silicate, a polymer, an acid, and surfactants. Surfactants may be provided to wet the surface of the bowl quickly and assist in cleaning performance. Non-limiting examples of suitable nonionic surfactants that may be used in an embodiment are as follows:

(1) The polyethylene oxide condensates of alkyl phenols. These compounds include the condensation products of alkyl phenols having an alkyl group with about 6 to 12 carbon atoms in either a straight chain or branched chain configuration with ethylene oxide, the ethylene oxide being present in an amount equal to 5 to 25 moles of ethylene oxide per mole of alkyl phenol. The alkyl substituent in such compounds may be derived, for example, from polymerized propylene, diisobutylene and the like. Examples of compounds of this type include nonyl phenol condensed with an average of 9.5 moles of ethylene oxide per mole of nonyl phenol; dodecylphenol condensed with an average of 12 moles of ethylene oxide per mole of phenol; dinonyl phenol condensed with an average of 15 moles of ethylene oxide per mole of phenol; and diisooctyl phenol condensed with an average of 15 moles of ethylene oxide per mole of phenol.

(2) The condensation products of aliphatic alcohols with about 1 to about 25 moles of ethylene oxide. The alkyl chain of the aliphatic alcohol may either be straight or branched, primary or secondary, with about 8 to about 22 carbon atoms. A 12-15 carbon alcohol may be ethoxylated with an average of 3-15 moles of ethylene oxide. Examples of such ethoxylated alcohols include, but are not limited to, the condensation product of myristyl alcohol condensed with an average of 10 moles of ethylene oxide per mole of alcohol; the condensation product of an average of 9 moles of ethylene oxide with coconut alcohol (a mixture of fatty alcohols with alkyl chains varying in length from about 10 to 14 carbon atoms); and the condensation product of undecanol with an average of about 7 moles of ethylene oxide.

(3) The condensation products of ethylene oxide with a hydrophobic base formed by the condensation of propylene oxide with propylene glycol. The hydrophobic portion of these compounds may have a molecular weight of from about 1500 to 1800 and exhibit water insolubility. The addition of polyoxyethylene moieties to this hydrophobic portion may increase the water solubility of the molecule as a whole, and the liquid character of the product may be retained up to the point where the polyoxyethylene content may be about 50% of the total weight of the condensation product, which corresponds to condensation with up to about 40 moles of ethylene oxide.

(4) The condensation of ethylene oxide with the product resulting from the reaction of propylene oxide and ethylenediamine. The hydrophobic moiety of these products include the reaction product of ethylenediamine and excess propylene oxide, the moiety having a molecular weight of from about 2500 to about 3000. This hydrophobic moiety may be condensed with propylene oxide to the extent that the condensation product contains from about 40% to about 80% by weight of polyoxyethylene and has a molecular weight of from about 5,000 to about 11,000.

(5) Amido amine oxide compounds. Examples of amido amine oxides, which may be useful in an embodiment, include, but are not limited to, cocoamidopropyl amine oxide, isostearylamidopropyl amine oxide, isostearylamidopropyl morpholine oxide, lauramidopropyl amine oxide, minkamidopropyl amine oxide, oleoamidopropyl amine oxide, olivamidopropyl amine oxide, sesamidopropyl amine oxide, stearamidopropyl amine oxide, wheat germ amidopropyl amine oxide, or mixtures thereof.

The nonionic surfactant may generally be present at a level of about 0.05% to about 50% by weight. The nonionic surfactant may be a 12-15 carbon alcohol, ethoxylated with an average of about 3-15 moles of ethylene oxide and may be about 1% of the cleaner.

Cationic surfactants may be used in an embodiment. Cationic surfactants include, but are not limited to, quaternary ammonium salts of the general formula:

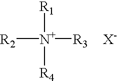

wherein the R groups coupled to the nitrogen are long or short hydrocarbon chains, typically alkyl, hydroxyalkyl or ethoxylated alkyl groups, and X is a solubilizing anion. Examples include, but are not limited to, coconut trimethyl ammonium chloride or bromide, coconut methyl dihydroxyethyl ammonium chloride or bromide, decyl triethyl ammonium chloride, decyl dimethyl hydroxyethyl ammonium chloride or bromide, C₁₂₋₁₅ dimethyl hydroxyethyl ammonium chloride or bromide, coconut dimethyl hydroxyethyl ammonium chloride or bromide, myristyl trimethyl ammonium methyl sulfate, lauryl dimethyl benzyl ammonium chloride or bromide, di-alkyl imidazolines, or mixtures thereof. The cationic surfactant may be present at a level of about 0.05% to about 50% by weight.

Anionic surfactants may be used in an embodiment. Examples of anionic surfactants include, but are not limited to, alkyl benzene sulfonates, having an alkyl chain length of C₈-C₁₅, primary and secondary alkyl sulfates such as C₈-C₁₅ primary alkyl sulfates, alkyl ether sulfates, olefin sulfonates; alkyl xylene sulfonates; dialkyl sulfosuccinates; fatty acid ester sulfonates, primary or secondary alcohol sulfates, wherein the linear or branched primary alcohol sulfates have 10 to 20 carbon atoms, or mixtures thereof. The anionic surfactants may be present at a level of about 0.05% to about 50% by weight. Additional surfactants may be found in U.S. Pat. No. 6,221,831 to Emery, et al. and U.S. Pat. No. 6,204,234 to Herbots, et al., both of which are incorporated herein by reference.

The acids include, but are not limited to, organic acids such as acetic, citric, lactic, tartaric acid, glycolic acid, or mixtures thereof. Other acids include, but are not limited to, hydrochloric acid and sulfamic acid. In an embodiment, a carboxylic acid may be a hydroxy monocarboxylic acid having up to 4 carbon atoms. In an embodiment, the acid content in the cleaner may be from about 1% to about 50% by weight of the formulation.

A synthetic layered silicate as described herein, may be added to the toilet bowl cleaner to enable the cleaner to cling to the sidewalls of the toilet bowl. Having the cleaner cling to the toilet bowl maximizes the time that the cleaner may be in contact with the lime scale and other deposits. If no thickener is used, the cleaner may run down the side of the toilet bowl too quickly, without effectively cleaning the surface.

Other ingredients such as pH adjusters, stabilizing agents, preservatives, fragrances and/or dyes may be included in the liquid cleaning composition. Stabilizing agents may be included to achieve phase stability, pH balance and other desired characteristics. Commonly used stabilizing agents include, but are not limited to, monoethanolamine, diethanolamine, triethanolamine, or mixtures thereof.

In an embodiment, the pH of the liquid cleaner may be in the range of about 1 to about 4. Agents for controlling the pH may be included. Examples include, but are not limited to, carbonates, bicarbonates, mono, di and triethanolamine, alkali metal hydroxides, or mixtures thereof.

Water may provide the balance of the liquid cleaning composition. In an embodiment, about 50% to about 99% water, by weight of the formulation, may be added. Fragrance may be added in an amount of up to about 1%. Further information on toilet bowl cleaners may be found in U.S. Pat. No. 6,153,572 to Stamm, which is incorporated herein by reference.

In an embodiment, a gel cleaner may be formulated with a synthetic layered silicate, as described herein, and with similar materials as in the toilet bowl cleaner. Other polymers and co-polymers may be added to increase the viscosity, such that the gel may remain stable when the gel is applied to a surface. U.S. Pat. No. 5,977,050 to Faris, which is incorporated herein by reference, provides additional information on gel cleaner formulations.

An oven cleaner may be formulated with a synthetic layered silicate as described herein. In an embodiment, water, synthetic layered silicate, polymer, solvent, alkali metal hydroxide, and tetrapotassium pyrophosphate may be mixed to make an oven cleaner. In an embodiment, about 2% of the synthetic layered silicate may be dispersed in sufficient water to provide a final water content, in the oven cleaner composition, of about 30% to about 50%. Tetrapotassium pyrophosphate may be added at about 0.05% to about 0.15%, by weight of water, in the oven cleaner composition. Alkali metal hydroxide may be added at about 10% to about 25%, by weight of water, in the oven cleaner composition. Other components may be added, such as, but not limited to, surfactants, solvents, and/or fragrances. Further formulations and details may be found in U.S. Pat. No. 3,779,933 to Eisen and U.S. Pat. No. 5,919,312 to Wierenga et al., both of which are incorporated herein by reference.

The synthetic layered silicate, as described herein, may be formulated into a toothpaste composition. In an embodiment, a toothpaste may be formulated to provide anti-caries and anti-plaque characteristics. Sorbitol, at about 25% to about 35%, by total weight of the formulation, may be added to water along with about 0.1% to about 0.5% synthetic layered silicate. The final amount of water in the formulation may be about 10% to about 50%, by weight of the formulation. Silica may be added to the formulation at about 10% to about 30% by weight of water, and the remainder of the formulation may include a fluoride compound for anti-caries activity, flavorings, anti-bacterial additives, and/or anti-plaque additives. The fluoride compounds include, but are not limited to, sodium fluoride, potassium fluoride, sodium monofluorophosphate, stannous fluoride, or mixtures thereof. Tetrasodium pyrophosphate may be added as an anti-plaque compound. Anti-bacterial toothpaste formulations include, but are not limited to, 2,4,4′-trichloro-2′-hydroxydiphenyl ether (Triclosan) or 2,2′-dihydroxy-5,5′-dibromo-diphenyl ether. Additional information on formulations may be found in U.S. Pat. No. 5,525,330 to Gaffar, et al., which is incorporated herein by reference.

A drilling fluid may be formulated with a synthetic layered silicate as described herein. In an embodiment, an aqueous calcium chloride solution, a synthetic layered silicate, a weighting agent (e.g. barium sulfate), and/or other viscosifiers, fluid loss agents, and/or surfactants may be used in the drilling fluid. Additional synthetic layered silicate may be added to the formulation if the drilling fluid contacts a salt formation. If the drilling fluid's ionic content increases, the additional layered synthetic silicate may be helpful in maintaining viscosity of the drilling fluid. Additional formulations and uses for synthetic layered silicates may be found in U.S. Pat. No 6,025,303 to Keilhofer, et al. and U.S. Pat. No. 6,022,833 to Mueller, et al., both of which are incorporated herein by reference.

A glass cleaner may be formulated with a synthetic layered silicate, as described herein, to produce a non-drip glass cleaner. In an embodiment, about 0.5% to about 1.5% synthetic layered silicate, by weight of the formulation, may be dispersed in water. About 2% to about 10% of an alcohol, by weight of the formulation, may be added to the synthetic layered silicate dispersion. Examples of an alcohol include, but are not limited to, methanol, ethanol, 1-propanol, isopropanol, butanol, or mixtures thereof. An oil emulsifier may be added at about 0.5% to about 10%, by weight of the formulation. Examples of oil emulsifiers include, but are not limited to, an ammonia solution, butoxyethanol, propylene glycol, ethylene glycol, ethylene glycol polymers, polyethylene, methoxypolyethylene glycols, or mixtures thereof. A surfactant may be added to the formulation at about 0.1% to about 1% by weight of the formulation. This formulation may provide a non-drip, streak-free composition for surface and glass cleaning. Other formulations may be found in U.S. Pat. No. 4,315,828 to Church and U.S. Pat. No. 5,798,324 to Svoboda, both of which are incorporated herein by reference.

A synthetic layered silicate, as described herein, may be formulated into a paint composition. In an embodiment, about 3 pounds to about 10 pounds of water may be added to about 55 pounds to about 65 pounds of resin. Examples of resins include, but are not limited to, (meth)acrylic acid, 2-hydroxyethyl (meth)acrylate, (meth) acrylic acid amideimides having a hydroxyl group, such as 1,1-dimethyl-1-(2-hydroxypropyl)amine (meth)acrylimide, 1,1-dimethyl-1-(2′-phenyl-2′-hydroxyethyl)amine (meth)acrylimide, 1,1-dimethyl-1-(2′-hydroxy-2′-phenoxypropyl)amine (meth) acrylimide, or mixtures thereof. About 2 pounds to about 5 pounds of propylene glycol, about 20 pounds to about 30 pounds of titanium dioxide, about 0.1 pounds to about 0.5 pounds of synthetic layered silicate may be added to the resin water mixture. In an embodiment, defoamers, dispersants and/or wetting agents may be added. Additional paint formulations may be found in U.S. Pat. No. 5,905,109 to Shimizu, et al., which is incorporated herein by reference.

A synthetic layered silicate, as described herein, may be formulated into a water-based printing ink composition. In an embodiment, a polymeric resin binder may be added to water, followed by the addition of a water-soluble resin binder to produce a water/binder mixture. A water-soluble rosin salt resin and an aqueous emulsion resin polymer may be added to the water/binder mixture. A rewetting agent, pigment, soybean oil, and the synthetic layered silicate may be added to the ink composition. Compositions for printing inks may be found in U.S. Pat. No. 6,200,372 to Krishnan, et al., which is incorporated herein by reference.

The following examples serve to illustrate methods of producing a synthetic layered silicate. The examples should not be considered limiting. Examples 1 and 2 are for comparative purposes and Examples 3, 4 and 5 are according to an embodiment.

The following tests were used in the examples to characterize the synthetic layered silicate material.

Electrolyte Tolerance

A 2.5% by weight dispersion of the synthetic layered silicate in demineralized water was prepared and left to stand for 24 hours. The dispersion was then diluted to 2% by weight synthetic layered silicate concentration with pre-determined aliquots of electrolyte and demineralized water. Viscosity measurements were taken after a total of 48 hours since first mixing. The effect of an electrolyte on the dispersion of the synthetic layered silicate may be related to the viscosity measurements. The viscosity measurements were conducted using a Brookfield DVII viscometer. The electrolyte tolerance of Examples 1-5 were determined using sodium sulfate as the electrolyte.

Hydration Rate

The rate of hydration of a 2% by weight dispersion of the synthetic layered silicate in demineralized water containing tetra-sodium pyrophosphate was assessed by measuring the time taken for the optical density of the dispersion to reduce to a specified value. The hydration rate may be defined as this time expressed in tenths of a minute. If the optical density had not reached the specified value after 20 minutes the hydration rate was expressed as the absorbance value at a wavelength of 550 nm using a 4 cm cell at that time.

Clarity

The optical density of a 2% by weight dispersion of the synthetic layered silicate in tetra-sodium pyrophosphate solution was measured at 550 nm using a 4 cm cell. The measurement was made 24 hours after the preparation of the dispersion; it was normally performed on the dispersion retained from the above Hydration Rate test.

Table 1 provides the quantities of reactants used in the preparation of Examples 1-5.

TABLE 1
Quantities of Reactants Used to Produce Synthetic Layered Silicate Materials

| | Example 1 | Example 2 | Example 3 | Example 4 | Example 5 |
|---|---|---|---|---|---|
| 1st amount of H₂O (g) | 168 | 168 | 279 | 330 | 379 |
| Li₂CO₃ (g) | 2.2 | 5.6 | 11.2 | 16.8 | 22.4 |
| 2nd amount of H₂O (g) | 301 | 269 | 269 | 269 | 269 |
| MgSO₄.7H₂O (g) | 209 | 187 | 187 | 187 | 187 |
| 3rd amount of H₂O (g) | 330 | 300 | 252 | 201 | 152 |
| Na₂CO₃(g) | 83 | 75 | 63 | 50 | 38 |
| Sodium Silicate solution^{a} (g) | 525^{a} | 525 | 525 | 525 | 525 |

^{a}14% by weight Na₂O (prepared by dilution of Crystal 79, supplied by Ineos Silicas, Warrington, UK)

TABLE 2
X-ray Fluorescence Analysis (% by weight oxide basis)

| | Example | Example | Example | Example | Example |
|---|---|---|---|---|---|
| | 1 | 2 | 3 | 4 | 5 |
| SiO₂ | 65.51 | 66.67 | 67.25 | 67.03 | 67.29 |
| MgO | 30.63 | 28.47 | 28.56 | 28.57 | 28.67 |
| Na₂O | 2.77 | 3.21 | 2.86 | 2.62 | 2.34 |
| SiO₂/MgO | 2.14 | 2.34 | 2.35 | 2.35 | 2.35 |

TABLE 3
Comparison of Lithium Carbonate Added as Reactant (in grams) and
Lithium Content in Final Product as % by weight Li

| | | Example | Example | Example | Example |
|---|---|---|---|---|---|
| | Example 1 | 2 | 3 | 4 | 5 |
| Lithium | 2.2 | 5.6 | 11.2 | 16.8 | 22.4 |
| Carbonate | | | | | |
| Lithium | 0.35 | 0.50 | 0.59 | 0.66 | 0.74 |

TABLE 4
Hydration Rate and Clarity of Product from Examples 2-5

| | Example 2 | Example 3 | Example 4 | Example 5 |
|---|---|---|---|---|
| Hydration rate | 1.35¹ | 0.25¹ | 175 | 0.79¹ |
| Clarity | 37.1 | 27.3 | 20.4 | 9.3 |

¹absorbance after 20 minutes

TABLE 5
Visacosity (cP) at 10 meq Na/g of Synthetic Layered Silicate

| | | Example | Example | Example | Example |
|---|---|---|---|---|---|
| | Example 1 | 2 | 3 | 4 | 5 |
| Viscosity | flocculated | 211000 | 108050 | 571000 | 182500 |
| (cP) | | | | | |

### EXAMPLE 1

Example 1 was prepared following the procedure in U.S. Pat. No. 4,049,780, Example 2, which is incorporated herein by reference.

### EXAMPLE 2

Example 2 was prepared using the following procedure and the quantities of reactants listed in Table 1. The Li₂CO₃, 5.6 g, was dispersed in 168 g of water with agitation for 1 hour at ambient temperature. Water (269 g) was added to the lithium carbonate solution, followed by addition of 187 g of MgSO₄.7H₂O. The magnesium sulfate was allowed to dissolve for a few minutes and then the solution was heated to 60° C. In a separate container, 75 g of Na₂CO₃ was dissolved in 300 g of water. The sodium carbonate solution was then added to the solution mixture of the lithium carbonate and magnesium sulfate over a period of 30 minutes, maintaining a temperature of 60° C. The sodium silicate solution (525 g) was added to the mixture of the magnesium sulfate, sodium carbonate and lithium carbonate over a period of 50 minutes, maintaining the temperature at 60° C. The resulting aqueous slurry was then heated to 98° C., and maintained at this temperature for 2 hours, condensing the vapor and returning the condensate to the aqueous slurry. The slurry was heated at 202° C. for 6 hours. The resulting hydrous magnesium silicate was separated by filtration, washed and dried. Example 2 represents a procedure in which the quantity of lithium compound used was only the stoichiometric amount needed to produce an atomic ratio equivalent to 8 atoms of silicon to 6 atoms of magnesium to 1 atom of lithium in the starting recipe.

### EXAMPLE 3

Example 3 was prepared following the procedure in Example 2, except that the reactant quantities were changed

### EXAMPLE 4

Example 4 was prepared following the procedure in Example 2, except that the reactant quantities were changed.

### EXAMPLE 5

Example 5 was prepared following the procedure in Example 2, except that the reactant quantities were changed.

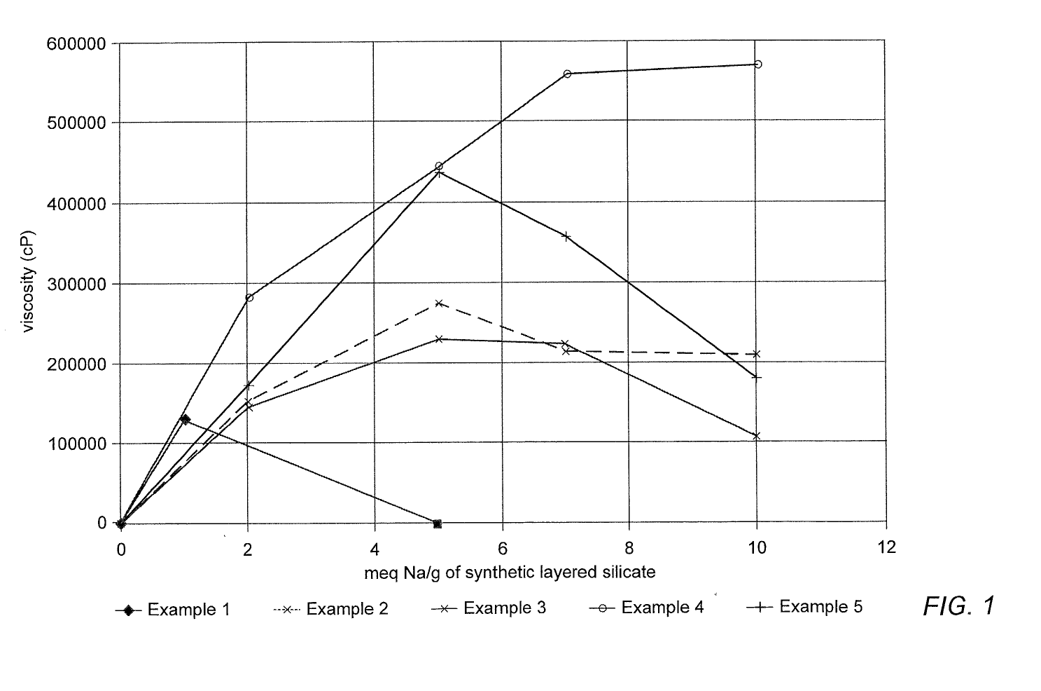

Referring to FIG. 1 and corresponding Table 5, Examples 3, 4 and 5 have a much greater tolerance to the presence of an electrolyte when compared to Example 1. Example 2 has improved tolerance to the presence of electrolyte, but this product has other disadvantages to Examples 3, 4 and 5. Table 2 is the oxide analysis of Examples 1-5. Table 3 is a comparison of the amount of lithium carbonate in the reactants compared to the amount of lithium present in Example 1-5. Table 4 indicates the Hydration Rate and Clarity of the products from Examples 2-5. The Hydration rate and Clarity data for Examples 3-5 indicate superior performance compared to Example 2.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

What is claimed is:

1. A synthetic layered silicate comprising the formula:
[Si₈(MgₐLi_{b})O₂₀(OH)_{4-y}F_{y}]^{z-}zM⁺
wherein a=4.75 to 5.45; b=0.25 to 1.25; y=0 to <4; z=12-2a-b; and M is Na⁺ or Li⁺; and
wherein the SiO₂/MgO is about 2.20 to about 2.40 and the lithium content is about 0.40% to about 0.80%; and,
wherein the synthetic layered silicate, when dispersed in an aqueous medium at about 2% by weight, wherein the aqueous medium contains from about 1 milliequivalent/gram synthetic layered silicate to about 12 milliequivalents/gram synthetic layered silicate of an electrolyte, increases the viscosity of the aqueous medium to greater than about 200,000 centipoise.

2. A method of making a synthetic layered silicate comprising:
preparing a magnesium metal compound solution, the magnesium metal compound solution comprising a magnesium cation;
preparing a carbonate compound solution, the carbonate compound solution comprising a carbonate anion;
mixing the magnesium metal compound solution and the carbonate compound solution;
adding a monovalent metal compound, and a silicate solution, to produce a synthetic layered silicate;
wherein the synthetic layered silicate, when dispersed in an aqueous medium at about 2% by weight, wherein the aqueous medium contains from about 1 milliequivalent/gram synthetic layered silicate to about 12 milliequivalents/gram synthetic layered silicate of an electrolyte, increases the viscosity of the aqueous medium to greater than about 200,000 centipoise.

3. The method of claim 2, wherein the carbonate compound comprises sodium carbonate.

4. The method of claim 2, wherein the monovalent metal compound comprises a lithium compound.

5. The method of claim 2, further comprising adding a monovalent halide compound.

6. The method of claim 5, wherein the monovalent halide compound comprises a fluoride compound.

7. The method of claim 2, wherein the silicate solution comprises sodium silicate.

8. The method of claim 2, wherein the silicate solution comprises silicic acid.

9. The method of claim 2, wherein the silicate solution comprises a mixture of silicon dioxide and sodium oxide.

10. The method of claim 2, wherein the silicate solution comprises sodium hexafluorosilicate.

11. The method of claim 2, wherein the carbonate solution is added to the divalent metal solution over a time period of greater than about 30 minutes.

12. The method of claim 2, wherein the reaction solutions are maintained at a temperature from about 40° C. to about 80° C.

13. The method of claim 2, wherein the solutions are stirred during reaction below about 1000 rpm.

14. The method of claim 2, further comprising adding the monovalent metal compound to the reaction mixture at about 100% to about 300% above the value of the monovalent metal content required to provide the cation of the synthetic layered silicate.

15. The method of claim 2, further comprising subjecting the synthetic layered silicate to a hydrothermal treatment.

16. The method of claim 15, wherein the hydrothermal treatment comprises heating the synthetic layered silicate to a temperature greater than about 100° C.

17. The method of claim 15, wherein the hydrothermal treatment comprises heating the synthetic layered silicate for greater than about 1 hour.

18. A synthetic layered silicate prepared by the process comprising:
preparing a magnesium metal compound solution, the magnesium metal compound solution comprising a magnesium cation;
preparing a carbonate compound solution, the carbonate compound solution comprising a carbonate anion;
mixing the magnesium metal compound solution and the carbonate compound solution;
adding a monovalent metal compound, and a silicate solution, to produce a synthetic layered silicate;
wherein the synthetic layered silicate, when dispersed in an aqueous medium at about 2% by weight, wherein the aqueous medium contains from about 1 milliequivalent/gram synthetic layered silicate to about 12 milliequivalents/gram synthetic layered silicate of an electrolyte, increases the viscosity of the aqueous medium to greater than about 200,000 centipoise.

19. The synthetic layered silicate product of claim 18, wherein the carbonate compound comprises sodium carbonate.

20. The synthetic layered silicate product of claim 18, wherein the monovalent metal compound comprises a lithium compound.

21. The synthetic layered silicate product of claim 18, further comprising adding a monovalent halide compound.

22. The synthetic layered silicate product of claim 21, wherein the monovalent halide compound comprises a fluoride compound.

23. The synthetic layered silicate product of claim 18, wherein the silicate solution comprises sodium silicate.

24. The synthetic layered silicate product of claim 18, wherein the silicate solution comprises silicic acid.

25. The synthetic layered silicate product of claim 18, wherein the silicate solution comprises a mixture of silicon dioxide and sodium oxide.

26. The synthetic layered silicate product of claim 18, wherein the silicate solution comprises sodium hexafluorosilicate.

27. The synthetic layered silicate product of claim 18, wherein the carbonate solution is added to the divalent metal solution over a time period of greater than about 30 minutes.

28. The synthetic layered silicate product of claim 18, wherein the reaction solutions are maintained at a temperature from about 40° C. to about 80° C.

29. The synthetic layered silicate product of claim 18, wherein the solutions are stirred during reaction below about 1000 rpm.

30. The synthetic layered silicate product of claim 18, further comprising adding the monovalent metal compound to the reaction mixture at about 100% to about 300% above the value of the monovalent metal content required to provide the cation of the synthetic layered silicate.

31. The synthetic layered silicate product of claim 18, further comprising subjecting the synthetic layered silicate to a hydrothermal treatment.

32. The synthetic layered silicate product of claim 31, wherein the hydrothermal treatment comprises heating the synthetic layered silicate to a temperature greater than about 100° C.

33. The synthetic layered silicate product of claim 31, wherein the hydrothermal treatment comprises heating the synthetic layered silicate for greater than about 1 hour.

34. A cleaner comprising:
water;
a synthetic layered silicate made by the process comprising:
preparing a magnesium metal compound solution, the magnesium metal compound solution comprising a magnesium cation;
preparing a carbonate compound solution, the carbonate compound solution comprising a carbonate anion;
mixing the magnesium metal compound solution and the carbonate compound solution;
adding a monovalent metal compound, and a silicate solution, to produce a synthetic layered silicate;
wherein the synthetic layered silicate, when dispersed in an aqueous medium at about 2% by weight, wherein the aqueous medium contains from about 1 milliequivalent/gram synthetic layered silicate to about 12 milliequivalents/gram synthetic layered silicate of an electrolyte, increases the viscosity of the aqueous medium to greater than about 200,000 centipoise; and,
a cleaner composition.

35. The synthetic layered silicate of claim 34, further comprising a monovalent halide compound.

36. The synthetic layered silicate of claim 35, wherein the monovalent halide compound comprises a fluoride compound.

37. The cleaner composition of claim 34 comprising:
a surfactant;
an acid;
stabilizing agents;
fragrances; and,
a dye.

38. The cleaner composition of claim 34 comprising:
a surfactant;
alkali hypochlorite;
fragrances; and,
a dye.

39. The cleaner composition of claim 34 comprising:
an alcohol;
an oil emulsifier; and,
aqueous ammonia.

40. An oven cleaner comprising:
water;
a synthetic layered silicate made by the process comprising:
preparing a magnesium metal compound solution, the magnesium metal compound solution comprising a magnesium cation;
preparing a carbonate compound solution, the carbonate compound solution comprising a carbonate anion;
mixing the magnesium metal compound solution and the carbonate compound solution;
adding a monovalent metal compound, and a silicate solution, to produce a synthetic layered silicate;
wherein the synthetic layered silicate, when dispersed in an aqueous medium at about 2% by weight, wherein the aqueous medium contains from about 1 milliequivalent/gram synthetic layered silicate to about 12 milliequivalents/gram synthetic layered silicate of an electrolyte, increases the viscosity of the aqueous medium to greater than about 200,000 centipoise;
an organic solvent;
an alkali metal hydroxide; and,
tetrapotassium pyrophosphate.

41. The synthetic layered silicate of claim 40, further comprising a monovalent halide compound.

42. The synthetic layered silicate of claim 41, wherein the monovalent halide compound comprises a fluoride compound.

43. A toothpaste comprising:
water;
sorbitol;
a synthetic layered silicate made by the process comprising:
preparing a magnesium metal compound solution, the magnesium metal compound solution comprising a magnesium cation;
preparing a carbonate compound solution, the carbonate compound solution comprising a carbonate anion;
mixing the magnesium metal compound solution and the carbonate compound solution;
adding a monovalent metal compound, and a silicate solution, to produce a synthetic layered silicate;
wherein the synthetic layered silicate, when dispersed in an aqueous medium at about 2% by weight, wherein the aqueous medium contains from about 1 milliequivalent/gram synthetic layered silicate to about 12 milliequivalents/gram synthetic layered silicate of an electrolyte, increases the viscosity of the aqueous medium to greater than about 200,000 centipoise;
silica; and,
an anti-caries compound.

44. The synthetic layered silicate of claim 43, further comprising a monovalent halide compound.

45. The synthetic layered silicate of claim 44, wherein the monovalent halide compound comprises a fluoride compound.

46. A drilling fluid comprising:
water;
a synthetic layered silicate made by the process comprising:
preparing a magnesium metal compound solution, the magnesium metal compound solution comprising a magnesium cation;
preparing a carbonate compound solution, the carbonate compound solution comprising a carbonate anion;
mixing the magnesium metal compound solution and the carbonate compound solution;
adding a monovalent metal compound, and a silicate solution, to produce a synthetic layered silicate;
wherein the synthetic layered silicate, when dispersed in an aqueous medium at about 2% by weight, wherein the aqueous medium contains from about 1 milliequivalent/gram synthetic layered silicate to about 12 milliequivalents/gram synthetic layered silicate of an electrolyte, increases the viscosity of the aqueous medium to greater than about 200,000 centipoise;
a weighting agent; and,
a fluid-loss agent.

47. The synthetic layered silicate of claim 46, further comprising a monovalent halide compound.

48. The synthetic layered silicate of claim 47, wherein the monovalent halide compound comprises a fluoride compound.

49. A paint comprising:
water;
propylene glycol;
titanium dioxide;
resin; and,
a synthetic layered silicate made by the process comprising:
preparing a magnesium metal compound solution, the magnesium metal compound solution comprising a magnesium cation;
preparing a carbonate compound solution, the carbonate compound solution comprising a carbonate anion;
mixing the magnesium metal compound solution and the carbonate compound solution;
adding a monovalent metal compound, and a silicate solution, to produce a synthetic layered silicate;
wherein the synthetic layered silicate, when dispersed in an aqueous medium at about 2% by weight, wherein the aqueous medium contains from about 1 milliequivalent/gram synthetic layered silicate to about 12 milliequivalents/gram synthetic layered silicate of an electrolyte, increases the viscosity of the aqueous medium to greater than about 200,000 centipoise.

50. The synthetic layered silicate of claim 49, further comprising a monovalent halide compound.

51. The synthetic layered silicate of claim 50, wherein the monovalent halide compound comprises a fluoride compound.

52. A printing ink comprising:
water;
a resin binder;
a rosin salt resin;
an aqueous emulsion resin polymer;
a rewetting agent;
a pigment;
a soybean oil; and,
a synthetic layered silicate made by the process comprising:
preparing a magnesium metal compound solution, the magnesium metal compound solution comprising a magnesium cation;
preparing a carbonate compound solution, the carbonate compound solution comprising a carbonate anion;
mixing the magnesium metal compound solution and the carbonate compound solution;
adding a monovalent metal compound, and a silicate solution, to produce a synthetic layered silicate;
wherein the synthetic layered silicate, when dispersed in an aqueous medium at about 2% by weight, wherein the aqueous medium contains from about 1 milliequivalent/gram synthetic layered silicate to about 12 milliequivalents/gram synthetic layered silicate of an electrolyte, increases the viscosity of the aqueous medium to greater than about 200,000 centipoise.

53. The synthetic layered silicate of claim 52, further comprising a monovalent halide compound.

54. The synthetic layered silicate of claim 53, wherein the monovalent halide compound comprises a fluoride compound.
